(19) United States
(12) Patent Application Publication
(10) Pub. No.: US 2026/0293892 A1
(43) Pub. Date: Oct. 1, 2026

(54) CYCLIZED BIOACTIVE PEPTIDES FOR CONTROLLING INSECTS

(71) Applicant: The United States of America, as represented by the Secretary of Agriculture, Washington, DC (US)

(72) Inventors: MAN Y. CHOI, ALBANY, OR (US); RYSSA K. PARKS, CORVALLIS, OR (US)

(21) Appl. No.: 19/709,109

(22) Filed: Jun. 16, 2026

Related U.S. Application Data
(63) Continuation-in-part of application No. 18/802,427, filed on Aug. 13, 2024.

(51) Int. Cl. A01N 37/46; A01N 25/00; A01N 25/02; A01P 7/04; C07K 7/64

(52) U.S. Cl. CPC A01N 37/46; A01N 25/006; A01N 25/02; A01P 7/04; C07K 7/64

(57) ABSTRACT

Disclosed are synthetic cyclized peptides developed from the western flower thrips (WFT) (Frankliniella occidentalis). The cyclized peptides disclosed can be used to repel, control, or deter Frankliniella spp., including Frankliniella occidentalis, and other insects from feeding on agricultural and horticultural plants. The cyclized peptides can be combined with bait materials, or applied directly to plants or areas. The peptides may also be produced by genetically modified plants or genetically engineered microbes, to express these peptides.

### CROSS-REFERENCE TO RELATED APPLICATIONS

This application is a continuation-in-part (CIP) of U.S. patent application Ser. No. 18/802,427, filed Aug. 13, 2024, entitled “BIOACTIVE PEPTIDES FOR CONTROLLING INSECTS”, which is hereby incorporated herein by reference in its entirety.

### INCORPORATION BY REFERENCE STATEMENT

An XML file for a “Sequence Listing XML”, submitted electronically using the USPTO Patent Center and having the file name: “Sequence_Listing-011425.xml”, creation date: Jun. 11, 2026, and file size: 23,952 bytes, is herein incorporated by reference.

### BACKGROUND OF THE INVENTION

### Field of Invention

Disclosed are synthetic cyclized peptides developed from the western flower thrips (Frankliniella occidentalis). Peptides disclosed can be used to repel, control, or deter Frankliniella spp., including Frankliniella occidentalis, and other insects from feeding on agricultural and horticultural plants. The peptides can be combined with bait materials, or applied directly to plants or areas. The peptides may also be produced by genetically modified plants engineered to express these peptides.

### Background

Frankliniella occidentalis (Pergande) (Thysanoptera: Thripidae), commonly known as the western flower thrips (WFT), is a globally significant pest in agriculture and horticulture often known for its relatively short life cycle and cryptic behavior, which allows it to hide in host plants, thereby contributing to its successful global dispersal via agricultural trade. Polyphagous to an alarming degree, it is a common sight for ornamental, greenhouse, and nursery industries, as well as for a variety of cropping systems. Currently, primary control method relies on chemical insecticides, including neonicotinoids, permethrins, carbamates, organophosphates, spinosyns, and avermectins, despite many adverse effects on the environment and increasing resistance. The current arsenal of tools to deal with WFT needs to be replaced with biological alternatives, or at least reduced in use. As alternative integrated pest management (IPM) tools, natural enemies such as mites and anthocorids, and semiochemicals such as attractants, are being used to control thrips, but are still not sufficient to control thrips populations in the field and in greenhouses.

The infestations and resulting damage/problems associated with thrips are increasing every year. Thrips have hundreds of host plants, including many ornamental and nursery crops. One of the most economically important pests is the western flower thrips (WFT), Frankliniella occidentalis, owing to its serious damage on greenhouse, horticultural and nursery crops. Not only direct damage from feeding on flowers and fruits, WFT also transmit tomato spotted wilt virus (TSWV) that is economically the most important. The combined value in 2022 of two of the most important vulnerable commodities i.e., greenhouse and nursery according to USDA and Oregon Department of Agriculture (ODA) is $1.4 billion in the Oregon alone. Those economic impacts are increasing every year. Today, WFT can be found on nearly all continents, making them one of the most economically significant pests, globally. Current control for thrips primarily relies on chemical insecticides despite causing potential negative effects to human health and environmental degradation as well as development of insecticide resistance.

Therefore, there is a strong need to develop environmentally friendly alternatives for WFT control. As presented herein, we have developed novel bioactive cyclized peptides that have broad applications. For example, the novel bioactive peptides in accordance with some embodiments of the present invention are commercially viable and specific for western flower thrips.

In insects, neuropeptides (NPs) and their cognate G protein-coupled receptors (GPCRs) regulate key physiological processes—including feeding, development, reproduction, and stress responses—through intracellular signaling pathways across life stages. Because NP signaling systems are essential for insect survival, they represent attractive molecular targets for pest management.

Insect NPs are part of a large group of neurohormones that regulate important biological functions and are found in invertebrates. A variety of peptide families from insects have been identified and classified by their core structures and functionalities. These neuropeptide ligands bind to GPCRs, a large group of signaling receptors for various signal transductions. GPCRs are membrane embedded proteins, also known as 7 transmembrane receptors, activated by a wide variety of stimulants including light, odorant molecules, peptide and non-peptide neurotransmitters, hormones, growth factors and lipids. They control a wide variety of physiological processes including sensory transduction, cell-cell communication, neuronal transmission, and hormonal signaling.

Among these NP signaling systems, the PRXamide peptide family is one of the most well-studied and promising groups. PRXamide peptides are characterized by a conserved C-terminal PRXamide motif (X=variable, i.e., any amino acid). The PRXamide family of neuropeptides is based on the core amino acid sequence at the C-terminal end that are required for activity and on sequence homology of their GPCRs. The PRXamide (PRX-NH2) family of neuropeptides are ubiquitously found in invertebrate animals. They are classified into four subgroups: pyrokinin (PK), pheromone biosynthesis activating neuropeptide (PBAN), diapause hormone (DH), capability CAPA (or CAPA-PVK), and ecdysis triggering hormone (ETH) throughout arthropods and other invertebrate. Numerous studies reported PRXamide-based peptide analogs, including sequence-modified variants and analogs conjugated to nonpeptidergic compounds, in efforts to develop peptide-based biopesticides. More recently, omics-based approaches have enabled the identification of bioactive molecules, including PRXamide peptide mimics, with activity against arthropod vectors. To screen such bioactive peptides and PRXamide mimics, including peptidomimetics, PRXamide GPCRs have been heterologously expressed in mammalian cells to identify receptor agonists and antagonists. Knowledge about the structure of specific peptide ligands and their receptors is necessary to more fully understand their interactions to facilitate the development of antagonists and agonists that can be developed for controlling potential biological targets.

The CAPA NPs are characterized, in most cases, by a C-terminal PRVamide or PRIamide motif. The first CAPA peptide, also known as CAPA-periviscerokinin (CAPA-PVK), a cardioacceleratory peptide, was isolated from the moth Manduca sexta. Subsequently, CAPA peptides became the accepted term, derived from the capability (capa) gene of Drosophila melanogaster. CAPA peptides are typically produced by neurosecretory cells in the insect central nervous system and released into the hemolymph. In insects, CAPA peptides have been implicated primarily in antidiuresis, cardioacceleration, and perivisceral muscle movement.

Like many insect NP receptors, CAPA receptors (CAPA-Rs) belong to Class A GPCRs and use calcium ions as second messengers. Although the physiological role of CAPA signaling in WFT remains unresolved, our previous studies characterized WFT CAPA peptides (Yun et al., “Identification and characterisation of PRXamide peptides in the western flower thrips, Frankliniella occidentalis”, Insect Molecular Biology, 32(6), 603-614 (2023). https://doi.org/10.1111/imb.12859) and their receptors (Parks et al., “Identification and Characterization of CAPA Receptor in Western Flower Thrips: Specific Insights Into CAPA Signaling by Naturally Occurring Peptides and CAPA-Derived Peptide Analogs”, Archives of Insect Biochemistry and Physiology, 120(4), e70116 (2025). https://doi.org/10.1002/arch.70116), including gene expression patterns, functional receptor expression, and downstream signaling pathways.

Notably, CAPA and CAPA-R expression levels were significantly higher in adult males than in females, which prompted our interest in the CAPA signaling system as a potential biological target for F. occidentalis management. Building on this foundation, as presented herein, we designed peptide analogs derived from native CAPA ligands and evaluated their insecticidal effects against F. occidentalis through in vitro and in vivo assays, together with 3D in silico modeling. Here, for example, in accordance with one or more aspects of the present disclosure, we identified nine cyclized peptide analogs with insecticidal activity that significantly reduced male survival, highlighting their potential as a new peptide-based strategy for thrips management. These molecules offer advantages such as biologically derived and environmentally degradable alternatives to conventional insecticides.

### SUMMARY OF THE INVENTION

Disclosed are synthetic cyclized peptides developed from the western flower thrips (Frankliniella occidentalis). Peptides disclosed can be used to repel, control, or deter Frankliniella spp., including Frankliniella occidentalis, and other insects from feeding on agricultural and horticultural plants. The peptides can be combined with bait materials, or applied directly to plants or areas. The peptides may also be produced by genetically modified plants engineered to express these peptides.

The novel bioactive peptides in accordance with some embodiments of the present invention will have potential benefits/advantages:
1. Bioactive peptides will not affect human health and environment.
2. The bioactive peptides can be mixed with conventional or biological insecticides to increase insecticidal efficacy.
3. Bioactive peptides by themselves or in combination with conventional insecticides will reduce the current chemical dose sprayed in the field.

Disclosed herein is an insecticide composition for controlling an insect, the composition comprising an effective amount of cyclized peptide having SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof. In one embodiment of the invention, the insecticide composition for controlling insects, comprises an effective amount of internally cyclized peptide having SEQ ID NO: 9, SEQ ID NO: 10, SEQ ID NO: 11, SEQ ID NO: 12, SEQ ID NO: 13, SEQ ID NO: 14, or a combination thereof. In another embodiment of the invention, the insecticide composition for controlling insects, comprises an effective amount of terminally cyclized peptide having SEQ ID NO: 15 SEQ ID NO: 16, SEQ ID NO: 17, SEQ ID NO: 18, SEQ ID NO: 19, SEQ ID NO: 20, or a combination thereof. In another embodiment of the invention, the insecticide composition further compromises an insecticide carrier. In one particular embodiment, the carrier is saline. In another embodiment of the invention, the insecticide composition further compromises one or more suitable propellants, carriers, diluents, adjuvants, preservatives, dispersants, solvents, or emulsifying agents.

Disclosed herein is a spray composition for controlling an insect, the spray comprises: (a) an effective amount of cyclized peptide having SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof and (b) a propellant.

Disclosed herein is a bait composition for controlling an insect, the bait comprises: (a) an effective amount of cyclized peptide having SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof, (b) one or more food materials; and (c) optionally a phagostimulant.

Disclosed herein is an insecticide composition for controlling Frankliniella ssp., the composition comprises of an effective amount of cyclized peptide having SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof. In one embodiment of the invention, the insecticide composition is for controlling Frankliniella occidentalis.

Also disclosed is a method for controlling an insect spp., the method comprises of contacting an insect or its environment with a biologically effective amount of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof, wherein the mortality of said insect increases. In one embodiment of the invention, the insect is Frankliniella occidentalis.

This summary is provided to introduce a selection of concepts in a simplified form that are further described below in the detailed description. This summary is not intended to identify key features of the claimed subject matter, nor is intended as an aid in determining the scope of the claimed invention.

### BRIEF DESCRIPTION OF THE DRAWINGS

Embodiments will hereinafter be described in conjunction with the appended drawings, where like designations denote like elements.

The patent or application file contains at least one drawing executed in color. Copies of this patent or patent application publication with color drawing(s) will be provided by the Office upon request and payment of the necessary fee.

FIG. 1 depicts peptide structures of the native ligands of western flower thrips, Frankliniella occidentalis (top), terminally cyclized analogs containing a disulfide bond at the peptide termini (middle), and internally cyclized analogs containing a disulfide bond at internal positions (bottom), according to one or more embodiments. All peptides are shown with amidated C-termini.

FIGS. 2A-2C depict activities of Frankliniella occidentalis CAPA-R to linear peptides (FIG. 2A), internally cyclized peptides (FIG. 2B), and terminally cyclized peptides (FIG. 2C) at 500 nM (upper) and 1 μM (lower), according to one or more embodiments. The native ligand QGLIPFPRV (CAPA2) (SEQ ID NO: 2) was run on each plate as a positive control to provide a normalization standard with which to compare fluorescence across plates. Asterisks denote statistical significance compared to the CAPA2 control for each treatment group (≥8 replicates), with each group analyzed by one-way ANOVA and Dunnett's correction; * <0.05, ** <0.01, *** <0.001, and **** <0.0001.

FIG. 3 depicts dose-response of Frankliniella occidentalis CAPA-R to internally cyclized peptides (CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14)), according to one or more embodiments. The native ligand, CAPA2 (QGLIPFPRV in FIG. 3), was used as a positive control. Terminally cyclized peptides were excluded from CAPA-R dose-response assays because they exhibited minimal activity at 500 nM and 1 μM concentrations (FIG. 2C). Data represent the mean±SEM of ≥8 replicates. EC50 values are shown in legend.

FIGS. 4A-4C depict dual-addition peptide assay: activities of Frankliniella occidentalis CAPA-R first challenged with linear peptides (FIG. 4A), internally cyclized analogs (FIG. 4B), or terminally cyclized analogs (FIG. 4C), according to one or more embodiments, followed by a second challenge with the native ligand CAPA2 (QGLIPFPRV) 1.5 minutes later. All peptides were applied at 500 nM. Asterisks denote statistical significance between the two peptide challenges (≥8 replicates) as determined by multiple paired t-tests with Holm-Šidák correction: * <0.05, ** <0.01, *** <0.001, and **** <0.0001.

FIG. 5 depicts activities of Frankliniella occidentalis CAPA-R in a dual-addition assay with extended incubation periods up to 60 minutes. Terminally cyclized analogs (1 μM), according to one or more embodiments, were applied first, followed by 500 nM CAPA2 (QGLIPFPRV) after 5, 15, 30, or 60 minutes. The “0 min” condition indicates that no peptide was applied prior to the addition of the native ligand. Different letters indicate p<0.05 for comparisons of the same peptide across incubation times, as determined by Tukey's HSD multiple comparisons test. Each dual-addition assay at each incubation duration was replicated eight times. Linear and internally cyclized analogs were excluded because most significantly activated CAPA-R (FIGS. 4A and 4B).

FIG. 6 depicts survival rates to determine the optimal sucrose concentration to use as a negative control in western flower thrips feeding assays. Treatments included 1-5% sucrose (w/v) and two negative controls: no water and 0% sucrose (water only). Survival distributions for each treatment were estimated using the nonparametric Kaplan-Meier method (PROC LIFETEST) in SAS 9.4. Survival was monitored for 7 days and compared using a log-rank test with Šidák-adjusted p-values. Survivors at the end of the trial were treated as censored observations. χ²=86.1230, df=3, p<0.0001. Letters denote statistically significant differences (≥10 replicates). The 1% and 5% sucrose solutions differed significantly from the 0% sucrose and “no water” treatments (Šidák-adjusted p<0.0002), but did not differ from each other (Šidák-adjusted p=1.0).

FIG. 7 depicts percent survival of female thrips fed various peptides, according to one or more embodiments, dissolved in 1% sucrose solution (pilot trial). Survivors at the end of the trial were treated as censored observations. Survival distributions for each treatment were estimated using the nonparametric Kaplan-Meier method (PROC LIFETEST) in SAS 9.4. Multiple survival curves were compared using a log rank test with Šidák adjusted p values. No significant differences were detected among the controls and peptide treatments (≥5 replicates).

FIGS. 8A-8D depict survival rates of Frankliniella occidentalis male adults fed linear and terminally cyclized peptides, according to one or more embodiments. Several preliminary trials were conducted using different combinations of linear and cyclized peptides on similar age males, collected from colonies on different days. Water and the nematode peptide FMRF (PGVLRF) (SEQ ID NO: 21) were used as negative controls, and a 1% sucrose solution served as the positive control. Survival distributions for each treatment were estimated using the nonparametric Kaplan-Meier method (PROC LIFETEST) in SAS 9.4. Multiple survival curves were compared using a log-rank test with Šidák-adjusted p-values. Different letters indicate statistically significant differences based on log-rank pairwise comparisons. In FIG. 8A, χ²=38.0402, df=6, p<0.0001 (≥5 replicates); in FIG. 8B, χ²=27.6847, df=6, p=0.0001 (>5 replicates); in FIG. 8C, χ²=49.4278, df=6, p<0.0001 (≥5 replicates); and in FIG. 8D, χ²=60.8600, df=5, p<0.0001 (≥10 replicates).

FIGS. 9A-9C depict schematic diagram of the feeding assay with Frankliniella occidentalis adults (FIG. 9A), and survival of male fed internally cyclized peptide analogs (FIG. 9B) or terminally cyclized peptide analogs (FIG. 9C) for 7 days, according to one or more embodiments. Thrips were individually placed in a 1.7-mL microcentrifuge tube containing a 1-μL droplet of 10 nmol peptide dissolved in a 1% sucrose solution. The 1% sucrose solution was used as a positive control. Water and the nematode FMRF-related peptide (PGVLRF), an unrelated neuropeptide, were used as negative controls. Distributions of survival times for each treatment were estimated using the nonparametric Kaplan-Meier method (PROC LIFETEST) in SAS 9.4. Multiple survival curves were compared using a log-rank test with Šidák-adjusted p<0.05. Different letters denote significant differences based on log-rank pairwise comparisons. Each treatment included at least fifteen or twenty-four replicates.

FIG. 10 depicts 3D models of Frankliniella occidentalis CAPA-R bound to the native ligand and terminally cyclized peptide analogs, according to one or more embodiments, generated using ColabFold and visualized in ChimeraX. Intracellular regions of the receptor models are obscured because the C-terminus was removed during modeling to ensure accurate localization of peptides to the extracellular region. Terminally cyclized peptides were selected for 3D modeling because terminally cyclized analogs showed neither agonistic nor antagonistic activity compared with linear and internally cyclized analogs (FIGS. 2A-2C & FIGS. 4A-4C).

FIG. 11 depicts residue pairs between the Frankliniella occidentalis CAPA-R and four different ligands, according to one or more embodiments, from which pseudobonds were identified between ligand and receptor residues limited to a distance of 3.0 Å between α-carbons. Pseudobonds PAE values were conditionally colored according to a color scale where 0=dark blue, 5=light blue, 10=yellow, 15=orange, and 20=red (lower numbers corresponding to higher confidence). Bolded and italicized residues indicate transmembrane status.

FIG. 12 depicts residue pairs between the Frankliniella occidentalis CAPA-R and all six internally cyclized peptides, according to one or more embodiments. Pseudobonds were identified between ligand and receptor residues limited to a distance of 3.0 Å between α-carbons. Pseudobonds PAE values were conditionally colored according to a color scale where 0=dark blue, 5=light blue, 10=yellow, 15=orange, and 20=red (lower numbers corresponding to higher confidence). Bolded and italicized residues indicate transmembrane status.

### BRIEF DESCRIPTION OF THE SEQUENCES

SEQ ID NO 1: is EVQGLFPFPRV, a synthetically generated peptide.

SEQ ID NO 2: is QGLIPFPRV, a synthetically generated peptide.

SEQ ID NO 3: is GLFPFPRV, a synthetically generated peptide.

SEQ ID NO 4: is LFPFPRV, a synthetically generated peptide.

SEQ ID NO 5: is FPFPRV, a synthetically generated peptide.

SEQ ID NO 6: is GLIPFPRV, a synthetically generated peptide.

SEQ ID NO 7: is LIPFPRV, a synthetically generated peptide.

SEQ ID NO 8: is IPFPRV, a synthetically generated peptide.

SEQ ID NO 9: is CLFPFCPRV, a synthetically generated peptide.

SEQ ID NO 10: is CLFPCFPRV, a synthetically generated peptide.

SEQ ID NO 11: is CLFCPFPRV, a synthetically generated peptide.

SEQ ID NO 12: is CLCFPFPRV, a synthetically generated peptide.

SEQ ID NO 13: is LCFCPFPRV, a synthetically generated peptide.

SEQ ID NO 14: is LFCPCFPRV, a synthetically generated peptide.

SEQ ID NO 15: is CGLFPFPRVC, a synthetically generated peptide.

SEQ ID NO 16: is CLFPFPRVC, a synthetically generated peptide.

SEQ ID NO 17: is CFPFPRVC, a synthetically generated peptide.

SEQ ID NO 18: is CGLIPFPRVC, a synthetically generated peptide.

SEQ ID NO 19: is CLIPFPRVC, a synthetically generated peptide.

SEQ ID NO 20: is CIPFPRVC, a synthetically generated peptide.

### DETAILED DESCRIPTION

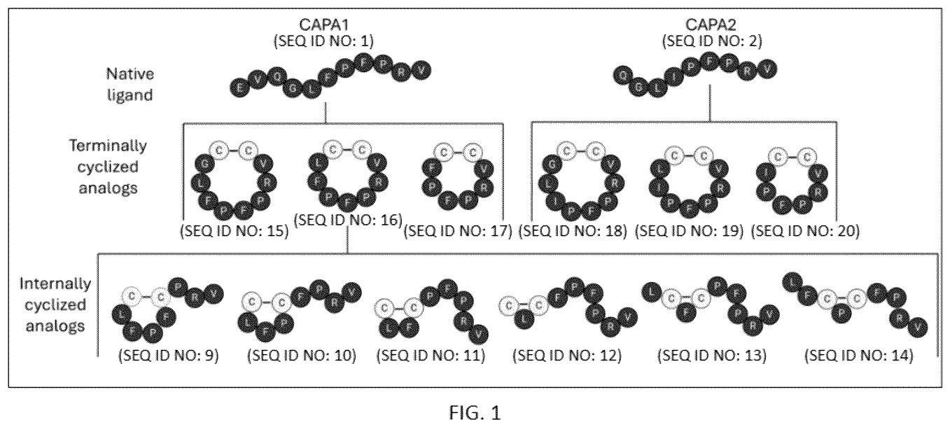

Western flower thrips, Frankliniella occidentalis, is a major agricultural pest that is difficult to manage because of its broad host range, cryptic behavior, rapid population growth, and increasing resistance to conventional insecticides. Insect neuropeptides and their cognate G protein-coupled receptors (GPCRs) have emerged as potential molecular targets for development of alternative pest management tools. As presented herein, we evaluated CAPA neuropeptide analogs for their ability to modulate signaling through the F. occidentalis CAPA receptor (CAPA-R). Twenty peptides (Table 1 and FIG. 1), including two natural ligands, six short linear peptides, six internally cyclized analogs, and six terminally cyclized analogs, were assessed using calcium mobilization assays in Sf9 cells expressing CAPA-R, dual-addition assays, oral feeding bioassays, and in silico receptor-ligand modeling. Most linear peptides and several internally cyclized peptides activated CAPA-R, whereas terminally cyclized peptides showed limited receptor activation in vitro. In feeding assays, nine cyclized peptides, including six internally cyclized and three terminally cyclized analogs, significantly reduced survival of adult male thrips. No significant effects on female survival were detected. Internally cyclized peptides generally showed greater activity in feeding assays than terminally cyclized peptides. Structural modeling further suggested that cyclized peptides interact within the CAPA-R ligand-binding region, although with orientations distinct from that of the native ligand. These findings indicate that cyclized CAPA peptide analogs can disrupt survival of male F. occidentalis and may provide a basis for development of peptide-based control strategies. More broadly, the results support insect neuropeptide GPCRs as promising targets for next-generation pest management approaches.

Neuropeptides are part of a large group of neurohormones that regulate important biological functions and are found in invertebrates. A variety of peptide families from western flower thrips (Frankliniella occidentalis), spotted-wing drosophila (Drosophila suzukii), diamondback moth (Plutella xylostella), western tarnished plant bug (Lygus Hesperus), and other insect neuropeptides have been identified and classified by their core structures and functionalities. These neuropeptide ligands bind to G protein-coupled receptors (GPCRs), a large group of signaling receptors for various signal transductions. GPCRs are membrane embedded proteins, also known as 7 transmembrane receptors, activated by a wide variety of stimulants including light, odorant molecules, peptide and non-peptide neurotransmitters, hormones, growth factors and lipids. They control a wide variety of physiological processes including sensory transduction, cell-cell communication, neuronal transmission, and hormonal signaling.

The PRXamide (X=variable, i.e., any amino acid) family of neuropeptides is based on the core amino acid sequence at the C-terminal end that are required for activity and on sequence homology of their GPCRs (Jurenka, R., Adv. Insect Physiol., (2015) 49: 123-70). The PRXamide family of neuropeptides are ubiquitous in invertebrate animals. The family includes proteins such as pyrokinin, pheromone biosynthesis-activating neuropeptide, diapause hormone, CAPA/periviscerokinin (a.k.a. cardioacceleratory peptide 2b), and ecdysis triggering hormone in many arthropods and gastropods. However, knowledge about structure of specific peptide ligands and their receptors is necessary to more fully understand their interactions to facilitate the development of antagonists and agonists.

Disclosed herein are synthetic cyclized peptides developed from the western flower thrips (Frankliniella occidentalis). Peptides disclosed can be used to repel, control, or deter Frankliniella spp., including Frankliniella occidentalis, and other insects from feeding on agricultural and horticultural plants. The peptides can be combined with bait materials, or applied directly to plants or areas. The peptides may also be produced by genetically modified plants engineered to express these peptides. In certain embodiments, the insects repelled, controlled, or deterred by the compositions or methods disclosed herein are Frankliniella spp., including Frankliniella occidentalis, as well as other insects such as, but not limited to, Drosophila spp., Plutella ssp., and Lygus ssp., including Drosophila suzukii, Plutella xylostella, and Lygus Hesperus.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2^{nd} ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount. For example, about 1.0 g means 0.9 g to 1.1 g and all values within that range, whether specifically stated or not.

“Optional” or “optionally” means that the subsequently described event or circumstance may or may not occur, and that the description includes instances in which said event or circumstance occurs and instances where it does not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences which are shorter or longer than the reference nucleic acid sequence, have different residues at particular positions, or a combination thereof.

“Activity” of a synthetic peptide, as used herein, refers to the capacity to obtain mortality or paralysis in target insects when such target insects are exposed to the peptides (e.g., via feeding or injection), which mortality or paralysis is significantly higher than a negative control (e.g., a buffer).

“Carrier” as used herein refers to any method of dispersal, dispensation, application, timed-release, encapsulation, microencapsulation, or the like to apply the insect-affecting composition as further described herein. In embodiments, such “carriers” may include a variety of microencapsulation, controlled-release, and other dispersion technologies available to those of ordinary skill in the art.

“Control” or “controlling” as used herein refers to any means for preventing infestation, reducing the population of already infested areas, or elimination of pest population(s) whose “control” is desired. Indeed, “controlling” as used herein refers to any indicia of success in prevention, elimination, reduction, repulsion, or amelioration of a pest population or pest problem.

An “effective amount” is an amount sufficient to effect desired beneficial or deleterious results. In terms of treatment, an “effective amount” is that amount sufficient to make the target pest non-functional by causing an adverse effect on that pest, including (but not limited to) physiological damage to the pest; inhibition or modulation of pest growth; inhibition or modulation of pest reproduction; or death of the pest. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The terms “polypeptide, peptide or protein” refer to polymers in which the monomers are amino acid residues (also abbreviated herein as “aa”) which are joined together through amide bonds. When the amino acids are alpha-amino acids, either the L-optical isomer or the D-optical isomer can be used. The terms are used interchangeably herein. These terms apply to amino acid polymers in which one or more amino acid residues are an artificial chemical mimetic of a corresponding naturally occurring amino acid, as well as to naturally occurring amino acid polymers and non-naturally occurring amino acid polymers.

A “conservative substitution” in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4 or 5 substitutions) retains the structure and function of the wild-type protein or peptide. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartate; or (d) a residue having a bulky side chain, for example, phenylalanine, is substituted for (or by) one not having a side chain, for example, glycine.

The term “phagostimulant” refers to any substance that will entice the target pest to ingest the selected bioactive peptide. Suitable phagostimulants include but are not limited to syrups, honey, aqueous solutions of sucrose, artificial sweeteners such as sucralose, saccharin, and other artificial sweeteners, starch, amino acids, and other proteins. Additionally, the bait material containing the bioactive peptide disclosed herein would be incorporated in water soluble baits, oil-in water or oil/water emulsion baits, liquid type or gel type of baits.

The ready-to-use preparations of phagostimulants can be in the form of a wettable powder, flowable concentrate solution, water soluble granules, ultra-low volume formulation, and the like, which can be applied to the target habitat. Phagostimulants can be used in combination with peptides of the present disclosure to enhance or encourage uptake by target pests. In essence, the combination is an insect bait. Such baits can also include any other component desired by one of skill in the art, such as carriers, preservatives, odorants, molluscicides, insecticides and the like. Phagostimulants can include carbohydrates such as glucose, fructose, arabinose, sorbitol, maltose, glucose, lactose, or any other small sugar. It will be obvious to a person skilled in the art that some carbohydrates and/or amino acids are likely to act as a deterrent. Thus, a bait, or other composition of the present invention can include phagostimulant(s) that attract a target pest and components that repel other animals (such as beneficial insects, pets and wildlife). Such variations are easily appreciated by any person skilled in the art.

The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman-Wunsch algorithm (Needleman and Wunsch, J Mol Biol, (1970) 48:3, 443-53). A computer-assisted sequence alignment can be conveniently performed using a standard software program such as GAP which is part of the Wisconsin Package Version 10.1 (Genetics Computer Group, Madison, Wis., USA) using the default scoring matrix with a gap creation penalty of 50 and a gap extension penalty of 3.

A “cyclized” peptide refers to a peptide in which a disulfide bond (S—S) forms a covalent linkage between the thiol (—SH) groups of two cysteine residues of the same chain (intramolecular) through oxidation. A two-electron oxidation process converts the two cysteine residues into cystine. When the two cysteine residues are respectively positioned at the N- and C-terminal ends of the peptide, we refer to the peptide as a “terminally cyclized peptide”. On the other hand, when at least one of the two cysteine residues is positioned internally within the peptide, we refer to peptide as an “internally cyclized peptide”. In laboratory synthesis, controlled oxidation (e.g., air oxidation, DMSO-assisted folding) can be used to form disulfide bonds in peptides. While cysteine residues in peptides can form disulfide bonds, this is not automatic—an oxidizing environment is required and, in some biological contexts, enzymatic assistance (e.g., in eukaryotic cells, disulfide bond formation is often catalyzed or assisted by enzymes such as protein disulfide isomerase (PDI) in the endoplasmic reticulum). In reducing environments, cysteines remain as free thiols, and disulfide bonds are not formed unless artificially oxidized.

### Peptides.

The peptides provided herein can be synthesized by any suitable method, such as exclusively solid-phase techniques, partial solid-phase techniques, fragment condensation, or classical solution addition. The amino acids of the compounds of the invention are typically joined to adjacent groups through amide linkages. For example, without being limited thereto, the peptide variants may be synthesized by methods well known to those skilled in the art of peptide synthesis, e.g., solution phase synthesis [see Finn and Hoffman, In “Proteins,” Vol. 2, 3rd Ed., H. Neurath and R. L. Hill (eds.), Academic Press, New York, pp. 105-253 (1976)], or solid phase synthesis [see Barany and Merrifield, In “The Peptides,” Vol. 2, E. Gross and J. Meienhofer (eds.), Academic Press, New York, pp. 3-284 (1979)], or stepwise solid phase synthesis as reported by Merrifield [J. Am. Chem. Soc. 85: 2149-2154 (1963)], the contents of each of which are incorporated herein by reference. However, the peptide fragments are preferably produced by recombinant DNA techniques, which are particularly suitable for large-scale use.

Synthesis by the use of recombinant DNA techniques, for the purpose of this application, should be understood to include the suitable employment of structural genes coding for the sequence as specified hereinafter. The synthetic peptides may also be obtained by transforming a microorganism or plant using an expression vector including a promoter or operator, or both, together with such structural genes and causing such transformed microorganisms or plant to express the peptide. For example, the synthetic peptides provided herein may be produced by genetically modified plants engineered to express these peptides. Accordingly, in accordance with some embodiments, the present invention extends to genetically modified plants or genetically engineered microbes, to produce these peptides.

Vectors used in practicing the present invention are selected to be operable as cloning vectors or expression vectors in the selected host cell. Numerous vectors are known to practitioners skilled in the art, and selection of an appropriate vector and host cell is a matter of choice. The vectors may, for example, be bacteriophage, plasmids, viruses, or hybrids thereof, such as those described in Sambrook et al. [Molecular Cloning: A Laboratory Manual, Cold Springs Harbor Laboratory, 1989] or Ausubel et al. [Current Protocols in Molecular Biology, John Wiley & Sons, Inc, 1995], the contents of each of which are herein incorporated by reference. Further, the vectors may be non-fusion vectors (i.e., those producing the peptides of the invention not fused to any heterologous polypeptide), or alternatively, fusion vectors (i.e., those producing the peptides fused to a vector encoded polypeptide). The fusion proteins would of course vary with the particular vector chosen.

Disruption and/or interference of the normal PRXamide peptide function would result in a variety of negative effects on insect survival and/or development. Some novel aspects and benefits/advantages of the compositions or methods disclosed herein are discussed below.

As presented herein, we evaluated CAPA neuropeptide analogs for their ability to modulate signaling through the F. occidentalis CAPA receptor (CAPA-R). Twenty peptides (Table 1, below), including two native ligands, six short linear peptides, six internally cyclized analogs, and six terminally cyclized analogs, were assessed using calcium mobilization assays in Sf9 cells expressing CAPA-R, dual-addition assays, oral feeding bioassays, and in silico receptor-ligand modeling. Table 1, below, shows the twenty peptides assessed.

TABLE 1
List of peptides used in the present disclosure

| | | Amino acid | |
|---|---|---|---|
| Ligand | Peptide | sequence* | SEQ ID NO: |
| Native | CAPA1 | EVQGLFPFPRV | SEQ ID NO: 1 |
| ligand | CAPA2 | QGLIPFPRV | SEQ ID NO: 2 |
| Linear | Peptides | GLFPFPRV | SEQ ID NO: 3 |
| peptide | derived | LFPFPRV | SEQ ID NO: 4 |
| | from CAPA1 | FPFPRV | SEQ ID NO: 5 |
| | Peptides | GLIPFPRV | SEQ ID NO: 6 |
| | derived | LIPFPRV | SEQ ID NO: 7 |
| | from CAPA2 | IPFPRV | SEQ ID NO: 8 |
| Cyclized | Internally | CLFPFCPRV | SEQ ID NO: 9 |
| peptide | cyclized | CLFPCFPRV | SEQ ID NO: 10 |
| | analogs | CLFCPFPRV | SEQ ID NO: 11 |
| | | CLCFPFPRV | SEQ ID NO: 12 |
| | | LCFCPFPRV | SEQ ID NO: 13 |
| | | LFCPCFPRV | SEQ ID NO: 14 |
| | Terminally | CGLFPFPRVC | SEQ ID NO: 15 |
| | cyclized | CLFPFPRVC | SEQ ID NO: 16 |
| | analogs | CFPFPRVC | SEQ ID NO: 17 |
| | | CGLIPFPRVC | SEQ ID NO: 18 |
| | | CLIPFPRVC | SEQ ID NO: 19 |
| | | CIPFPRVC | SEQ ID NO: 20 |
| Non-insect | MelinFMRF | PGVLRF | SEQ ID NO: 21 |
| peptide | | | |

*All C-terminal ends of the peptides are amidated (NH₂). MelinFMRF (SEQ ID NO: 21) is from a nematode species (Meloidogyne incognita).

FIG. 1 depicts peptide structures of the two native ligands (top), the six terminally cyclized analogs containing a disulfide bond at the peptide termini (middle), and the six internally cyclized analogs containing a disulfide bond at internal positions (bottom), according to one or more embodiments. All peptides are shown with amidated C-termini.

Knowledge of the structure of the novel peptide agonists facilitates intelligent synthesis of chemical structures with a similar functional group conformation, but with better physical/physiological properties.

Thus, the compositions and methods disclosed herein will have general applicability. The ubiquitous PRXamide GPCRs play a key role in normal development as well as critical adult activities, thus the compositions and methods disclosed herein are extremely versatile as the bioactive peptides negatively affect the normal functioning of both immature and adult insects.

### MATERIALS AND METHODS

### Insects and Peptides.

The WFT colony was established from a wild population collected on alyssum flowers and identified as Frankliniella occidentalis via DNA barcoding and was maintained in accordance with previously established methods. Thrips were reared on cotyledons of Shirofumi soybeans (Glycine max) or red kidney beans (Phaseolus vulgaris) (Fedco Seeds, Clinton, ME, USA), under a L:D=16:8, 60% RH, and 25±1° C., in the USDA-ARS, Corvallis, OR, USA.

All peptides disclosed herein (Table 1) were designed with a C-terminal amide group (—NH₂) and synthesized at >95% purity (Peptide 2.0, Chantilly, VA, USA). Cyclized peptides were generated by introducing two cysteine residues to form a disulfide bridge, either at internal positions or at the terminal ends of the peptides. When at least one cysteine is located within the peptide sequence, we refer to it as an internally cyclized peptide. When both cysteines are positioned at the N- and C-terminal ends, we refer to it as a terminally cyclized peptide (also called a circular peptide) (Table 1 and FIG. 1). Synthetic peptides were resuspended in water, aliquoted into working stocks, lyophilized, and stored at −20° C. until use.

### Functional Expression of CAPA-R and Measuring Ca²⁺ Mobilization.

The activity of F. occidentalis CAPA-R (FraocCAPA-R) functionally expressed in Sf9 cells was measured using calcium mobilization, quantified as fluorescence intensity with a FLIPR Calcium Assay system (Molecular Devices, San Jose, CA, USA) operated on a FlexStation 3, as described previously (Parks et al., 2025).

Measurement of Agonistic and Antagonist Effect with CAPA Analogs.

FraocCAPA-R was initially tested with 500 nM or 1 μM of eighteen short peptides, comprising both linear and cyclized analogs. CAPA2 served as the positive control because the native ligand exhibits stronger activation (Parks et al., 2025).

Two dual-addition assays were conducted to investigate the agonistic and/or antagonistic effects of the eighteen short peptides. In these assays, the receptor is first challenged with the compound of interest, followed by addition of the native ligand. For the first dual-addition assay, each peptide (500 nM) was challenged first and incubated for 1.5 min when it is a full period for the peptide to activate CAPA-R for measuring the fluorescent intensity. Then, the same dose of the native ligand CAPA2 was introduced and followed the change in the fluorescent intensities was measured for another 1.5 min. The program ran for 4 min total, after which data were sorted in Microsoft Excel and the level of activation from peptide exposure was normalized relative to the maximum fluorescence value obtained from the peptides was normalized relative to initial baseline fluorescence.

The second dual assay was conducted with longer incubations to examine whether CAPA-R could resensitize with extended recovery time; therefore, various incubation periods were also tested. In these assays, 1 μM of a modified peptide was added manually, followed by 500 nM CAPA2 after 5, 15, 30, or 60 min. Each dual-addition assay at each incubation duration was replicated eight times.

### Dose Response of CAPA-R to Cyclized Peptides

Six cyclized peptides containing internal disulfide bonds were evaluated for their binding activities on CAPA-R using three-fold serial dilutions ranging from 0.08 nM to 1500 nM, with eight replicates per concentration. Half-maximal effective concentration (EC₅₀) values were determined using the sum-of-squares F-test in GraphPad Prism version 7.0 (San Diego, CA), as described previously (Parks et al., 2025). Low EC₅₀ values are an indication of strong peptide-receptor binding.

### Thrips Feeding Assay

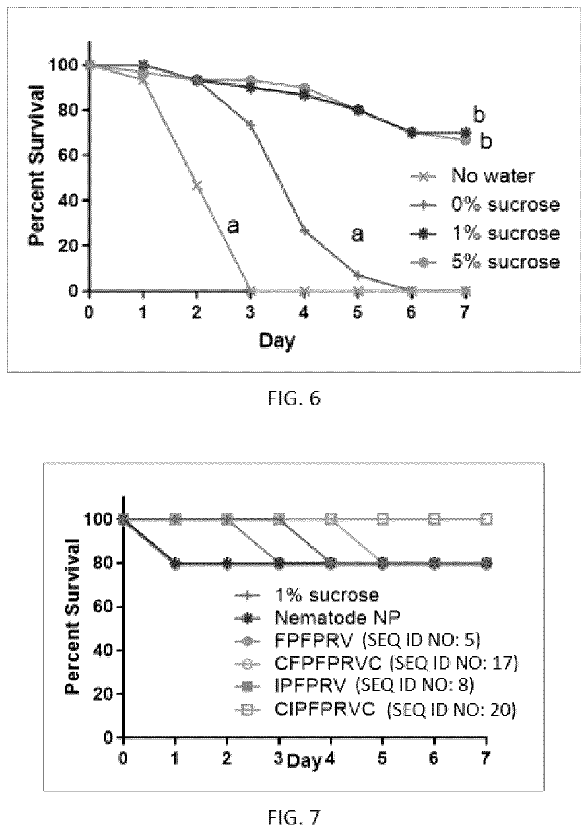

Two- to four-day-old WFT adults were individually placed into a 1.7 mL microcentrifuge tube containing a 1 μL droplet of 10 nmol peptide dissolved in a 1 percent sucrose solution, which had been confirmed as the optimum sugar concentration. (FIG. 6).

The droplet was replenished as needed, typically on days 3-5, after which further replenishment was not required. Four trials were conducted, consisting of 15 individuals per treatment for a total of seven treatments, and 24-25 individuals per treatment for a total of ten treatments. Mortality was monitored for seven days. A 1% sucrose solution and MelinFMRF (PGVLRFamide) from the nematode (Meloidogyne incognita) dissolved in a 1% sucrose solution, were used as positive controls, and water alone was used as a negative control.

Survival rates were analyzed by the nonparametric Kaplan-Meier Proc Lifetest in SAS 9.4 (SAS Institute, Cary, NC, USA). Survival rates were compared using a log-rank test and corrected using an adjusted Šidák P-value. Any remaining survivors past the last day of recording mortality were censored for analysis.

### 3D Modeling of CAPA-R-Ligand Complex

3D models of extracellular and transmembrane domains of F. occidentalis CAPA-R were predicted in ChimeraX using ColabFold v1.5.3: CAPA-R in complex with the native ligand, CAPA2; and CAPA-R in complex with the three terminally cyclized peptides selected because they significantly reduced thrips survival after feeding assays. To localize docking possibilities of the ligand to the N-terminus and transmembrane domain only, the C-terminal sequence was omitted from the input. Five different models of each receptor-ligand complex were generated from the predictive algorithm, which overall showed consensus regarding the overall binding area of both native and modified ligands. Once the best model was selected, the predicted aligned error (PAE) of each receptor-ligand complex was assessed. PAE rates the level of confidence of protein domain proximities on a scale of 0-30, 0 being most confident and 30 being least confident.

### EXAMPLES

### Example 1—Functional Expression of CAPA-R and Challenge with CAPA Analogs

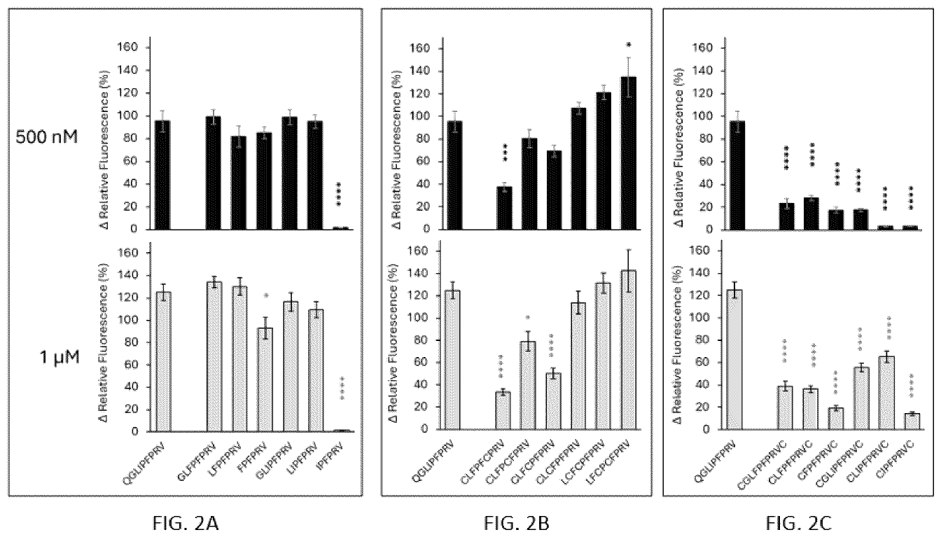

As shown in FIGS. 2A-2C, receptor activation was evaluated using nineteen peptides, including the native ligand CAPA2 (QGLIPFPRV in FIGS. 2A-2C) as well as modified (6-8 amino acids) peptides—six linear peptides (i.e., as shown in FIG. 2A, GLFPFPRV (SEQ ID NO: 3), LFPFPRV (SEQ ID NO: 4), FPFPRV (SEQ ID NO: 5), GLIPFPRV (SEQ ID NO: 6), LIPFPRV (SEQ ID NO: 7), and IPFPRV (SEQ ID NO: 8)) and twelve cyclized peptides (i.e., as shown in FIG. 2B, six internally cyclized peptides, i.e., CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14); and as shown in FIG. 2C, six terminally cyclized peptides, i.e., CGLFPFPRVC (SEQ ID NO: 15), CLFPFPRVC (SEQ ID NO: 16), CFPFPRVC (SEQ ID NO: 17), CGLIPFPRVC, (SEQ ID NO: 18), CLIPFPRVC (SEQ ID NO: 19), and CIPFPRVC (SEQ ID NO: 20)), tested at 500 nM and 1 μM concentrations.

Referring now to FIG. 2A, the linear shortened peptides showed strong activation of the receptor at both concentrations, and their activation levels were not statistically different from those of CAPA2, except for IPFPRV (SEQ ID NO: 8). Water, used as the negative control, did not activate the receptor.

Referring now to FIGS. 2B and 2C, twelve cyclized peptides containing two cysteines that form a disulfide bond either internally (FIG. 2B) or at the terminal sites (FIG. 2C) of the peptide sequence were tested at both concentrations. As shown in FIG. 2B, among the six internally cyclized peptides (i.e., CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14)), only CLFPFCPRV (SEQ ID NO: 9) showed significantly reduced receptor activation at both concentrations. In contrast, CLCFPFPRV (SEQ ID NO: 12), LCFCPFPRV (SEQ ID NO: 13), and LFCPCFPRV (SEQ ID NO: 14) strongly activated the receptor, with activation levels comparable to those of their corresponding linear peptides.

However, as shown in FIG. 2C, all six terminally cyclized peptides (i.e., CGLFPFPRVC (SEQ ID NO: 15), CLFPFPRVC (SEQ ID NO: 16), CFPFPRVC (SEQ ID NO: 17), CGLIPFPRVC (SEQ ID NO: 18), CLIPFPRVC (SEQ ID NO: 19), and CIPFPRVC (SEQ ID NO: 20)) failed to activate the receptor at either concentration. These results highlight a marked difference in activation profiles between internally and terminally cyclized peptides.

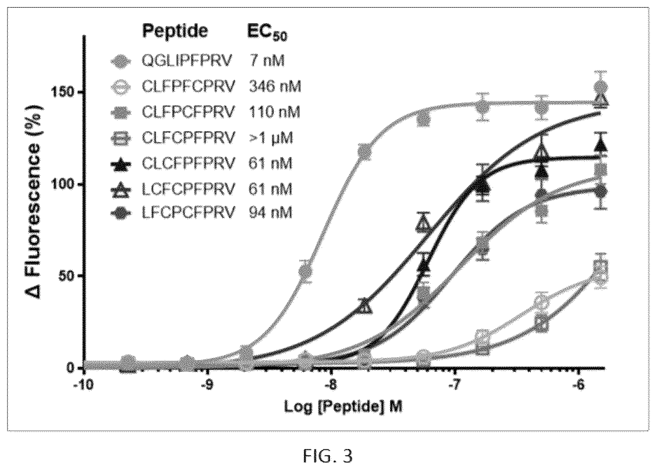

Based on the activation results from the two high-dose assays, six internally cyclized peptides (i.e., CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14)) were selected for further evaluation to determine their binding affinity to the receptor. As shown in FIG. 3, the EC₅₀ values of four cyclized peptides (LCFCPFPRV (SEQ ID NO: 13), CLCFPFPRV (SEQ ID NO: 12), LFCPCFPRV (SEQ ID NO: 14), and CLFPCFPRV (SEQ NO ID 10)) ranged from 61 nM to 110 nM, indicating that these peptides clearly activated the receptor, although their binding activities remained lower than that of the native ligand CAPA2 (QGLIPFPRV in in FIG. 3). In contrast, as also shown in FIG. 3, the remaining two cyclized peptides, CLFPFCPRV (SEQ ID NO: 9) and CLFCPFPRV (SEQ ID NO: 11), were substantially higher EC₅₀ values—310 nM and >1 μM, respectively—indicating much weaker receptor affinity.

Linear peptides were excluded because they were thoroughly characterized in our previous study (Parks et al., 2025), and terminally cyclized peptides were omitted due to their poor receptor activation (FIG. 2C).

### Example 2—Agonistic and Antagonistic Effect with CAPA Analogs

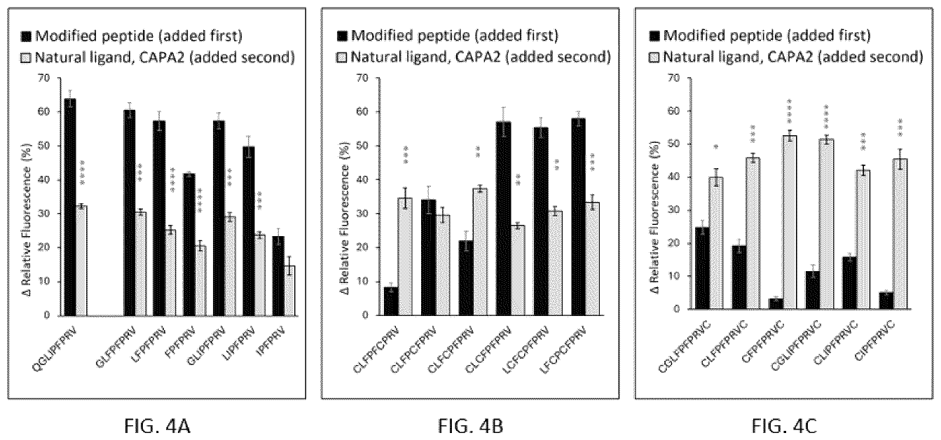

As shown in FIGS. 4A-4C, to evaluate agonistic or antagonistic effects of the linear and cyclized peptides, a dual-addition assay was conducted with eighteen short peptides.

Referring now to FIG. 4A, a first trial with six linear peptides showed that the activation and suppression levels through the five peptide analogs, GLFPFPRV (SEQ ID NO: 3), LFPFPRV (SEQ ID NO: 4), FPFPRV (SEQ ID NO: 5), GLIPFPRV (SEQ ID NO: 6), and LIPFPRV (SEQ ID NO: 7), were similar trends to those of the native ligand CAPA2 (QGLIPFPRV in FIG. 4A). Therefore, these peptides seem to have an agonistic effect. However, IPFPRV (SEQ ID NO: 8) showed significantly lower activate CAPA-R at before and after the peptide challenge, indicating IPFPRV (SEQ ID NO: 3) neither activates CAPA-R nor allows CAPA2 (QGLIPFPRV in FIG. 4A) to activate the receptor, suggesting that it acts not as an agonist but rather as an antagonist.

Referring now to FIG. 4B, the six internally cyclized peptides exhibited mixed effects on CAPA-R. The three cyclized peptides (CLCFPFPRV (SEQ ID NO: 12), LCFCPFPRV (SEQ ID NO: 13), and LFCPCFPRV (SEQ ID NO: 14)) significantly activated CAPA-R, but they also inhibited CAPA-R activation by CAPA2 after the subsequent incubation with the peptides.

Referring now to FIG. 4C, the six terminally cyclized peptides (CGLFPFPRVC (SEQ ID NO: 15), CLFPFPRVC (SEQ ID NO: 16), CFPFPRVC (SEQ ID NO: 17), CGLIPFPRVC (SEQ ID NO: 18), CLIPFPRVC (SEQ ID NO: 19), and CIPFPRVC (SEQ ID NO: 20)) showed little or no activation of CAPA R during the initial 1.5 min incubation and did not affect CAPA2 induced activation for the subsequent incubation. These results indicate that although some terminally cyclized peptides exhibit weak agonistic activity, none display antagonistic effects on CAPA-R. We conducted with the incubation time gradually from 1.5 min to 2.5 min and observed no changes in these overall trends (data not shown).

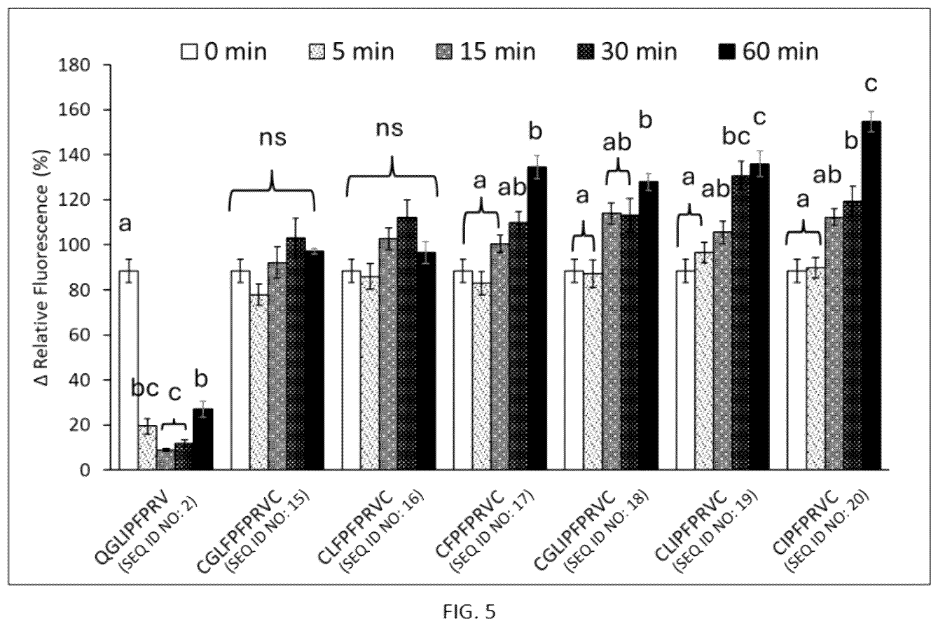

Since the terminally cyclized peptides exhibited neither agonistic nor antagonistic effects during the shorter incubation periods, we conducted a second trial to assess whether extended incubations with cyclized peptides influence the binding affinity between CAPA2 and CAPA-R. In the second trial, the terminally cyclized peptides were incubated for 5-60 min prior to CAPA2 challenge. As shown in FIG. 5, across all incubation periods, these terminally cyclized peptides did not reduce CAPA2 binding activity. However, the native ligand CAPA2 (QGLIPFPRV in FIG. 5) used as a control significantly decreased receptor binding affinity following prolonged incubation. Notably, as shown in FIG. 5, the two shortest peptides, CFPFPRVC (SEQ ID NO: 17) and CIPFPRVC (SEQ ID NO: 20), produced a significant increase in CAPA-R binding affinity with CAPA2 after 60 min of incubation. The changes in binding affinity observed for these peptides were consistent with the patterns measured in the short intervals (1.5-2.5 min) of the dual-addition assay (FIG. 4C). Together, these results indicate that short peptides containing terminal cyclization may enhance CAPA-R interaction after extended exposure conditions.

### Example 3—Thrips Feeding on Peptides

For the feeding assays, adult thrips were provided with a 1 μL water droplet containing 10 nmol peptide and 1% sucrose for 7 days. As shown in FIG. 6, the preliminary result showed that thrips survival rates were not significantly different among thrips fed on 1-5% sucrose solutions for the 7 days.

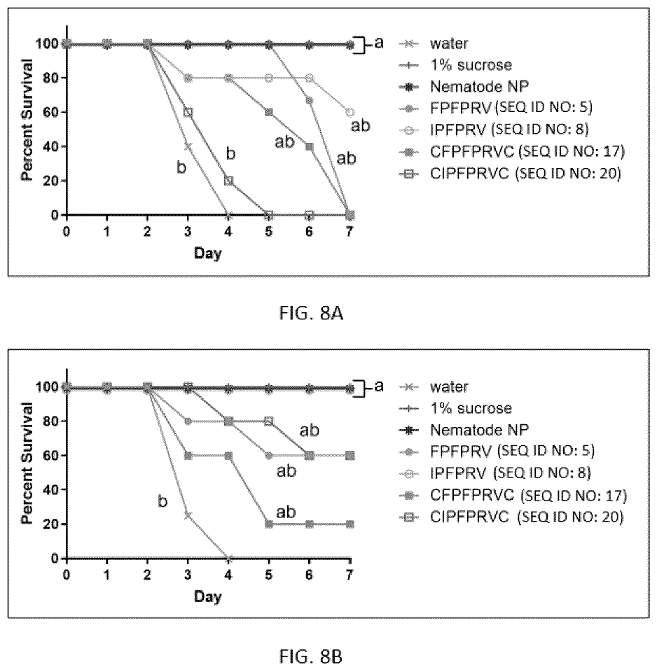

As shown in FIG. 7, we observed that female WFT did not exhibit statistically significant mortality following peptide feeding. Our previous studies demonstrated that the expression levels of capa mRNA (Yun et al., 2023) and capa-r mRNA (Parks et al., 2025) are approximately 10-fold and 6-fold higher, respectively, in adult males compared with adult females. Therefore, we focused our feeding assays on adult males. As preliminary feeding tests, short and cyclized peptides were administered to adults to monitor mortality and identify peptides with potential biological activity.

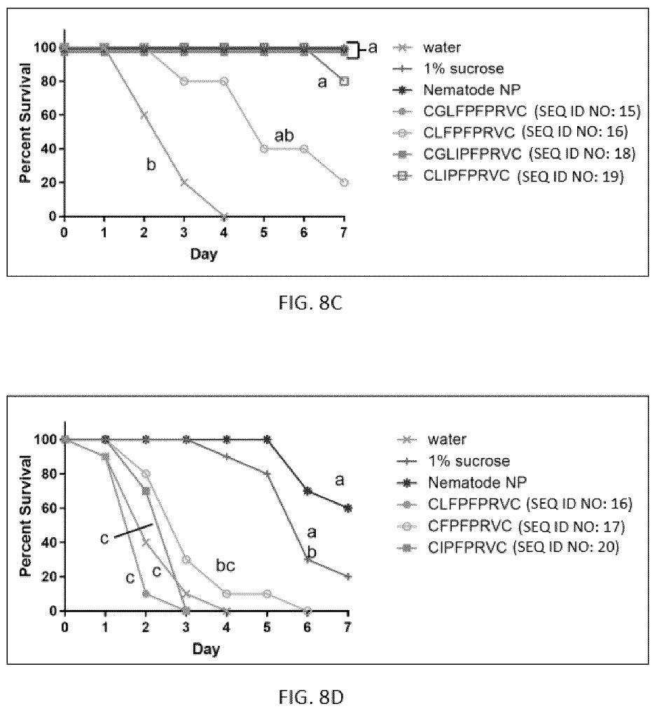

As shown in FIGS. 8A-8D, as preliminary tests, short peptides (i.e., FPFPRV (SEQ ID NO: 5) and IPFPRV (SEQ ID NO: 8)) and terminally cyclized peptides (i.e., CFPFPRVC (SEQ ID NO: 17), CIPFPRVC (SEQ ID NO: 20), CGLFPFPRVC (SEQ ID NO: 15), CLFPFPRVC (SEQ ID NO: 16), CGLIPFPRVC (SEQ ID NO: 18), CLIPFPRVC (SEQ ID NO: 19)) were fed on male adult thrips, their mortalities were observed from over twenty replications. As positive controls, a 1% sucrose solution and a non-insect neuropeptide from the nematode dissolved in 1% sucrose solution were used, and a negative control—water only—was included.

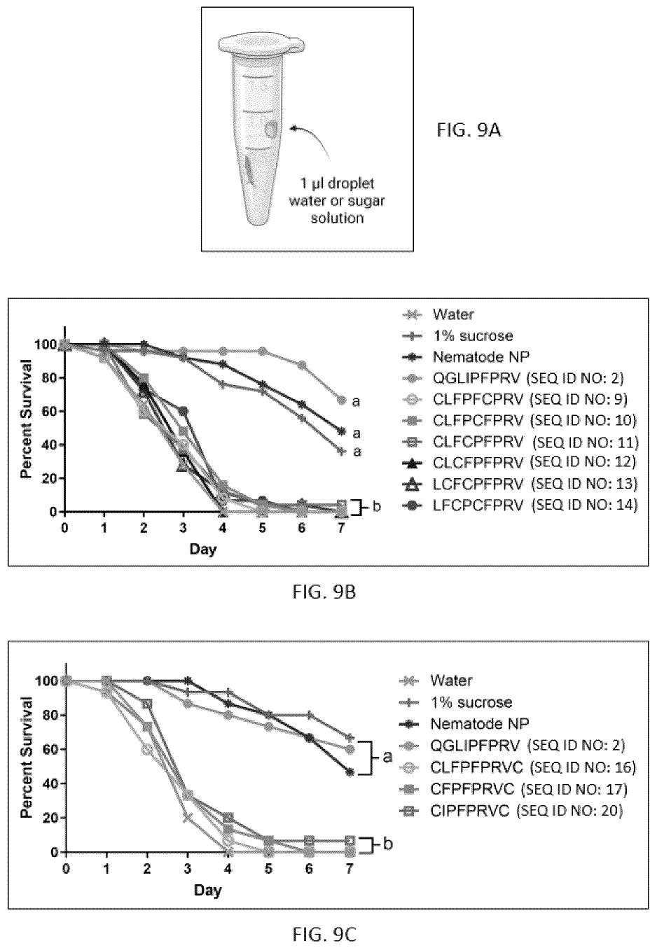

FIGS. 9A, 9B, and 9C respectively depict a feeding assay with bioactive peptides on thrips (FIG. 9A), percentage survival of male thrips fed six internally cyclized peptide analogs (FIG. 9B), and percentage survival of male thrips fed three selected terminally cyclized peptide analogs (FIG. 9C) for seven days. Thrips were individually placed in a 1.7-mL microcentrifuge tube containing a 1-μL droplet of 10 nmol peptide dissolved in a 1% sucrose solution. A 1% sucrose solution and the nematode FMRF peptide (PGVLRF) dissolved in a 1% sucrose solution, were used as positive controls. Water alone was used as a negative control.

As shown in FIG. 9C, thrips fed on any of the three selected terminally cyclized peptides (CLFPFPRVC (SEQ ID NO: 16), CFPFPRVC (SEQ ID NO: 17), and CIPFPRVC (SEQ ID NO: 20)) exhibited significantly higher mortality compared with the two positive controls—1% sucrose and the nematode neuropeptide (NP), a non-insect neuropeptide (Šidák-adjusted P<0.05). Survival of peptide-fed thrips, and thrips provided only water, declined to 20% or lower after four days of feeding. In contrast, survival of the positive controls and CAPA2-fed thrips (QGLIPFPRV in FIG. 9C) remained at 80% or higher over the same period. Overall, 7-day survivorship of fifteen F. occidentalis adult males treated with the three cyclized peptides differed significantly from the control treatments (χ²=77.74, df=6, p<0.0001).

As shown in FIG. 9B, thrips fed on any of the six internally cyclized peptides (CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14)) exhibited significantly higher mortality than the two positive controls, 1% sucrose and the nematode NP (Šidák-adjusted P<0.05). Similarly, thrips fed on the six terminally cyclized peptides (FIG. 9B) and the three selected terminally cyclized peptides (CLFPFPRVC (SEQ ID NO: 16), CFPFPRVC (SEQ ID NO: 17), and CIPFPRVC (SEQ ID NO: 20)) (FIG. 9C) also exhibited significantly higher mortality than those fed the two positive controls (Šidák-adjusted P<0.05).

Survival of peptide-fed thrips, and thrips provided only water, declined to 15% or lower after four days of feeding. In contrast, survival of the positive controls and CAPA2-fed thrips (QGLIPFPRV in FIGS. 9B and 9C) remained at 75% or higher over the same period. Overall, 7-day survivorship of twenty-five F. occidentalis adult males treated with the nine cyclized peptides differed significantly from the control treatments (χ²=154.6063, df=9, p<0.0001). Taken together, comparison of the two feeding trials indicates that internally cyclized peptides were more effective at reducing thrips survival than terminally cyclized peptides.

### Example 4—3D Model Prediction of the Receptor-Ligand Complex

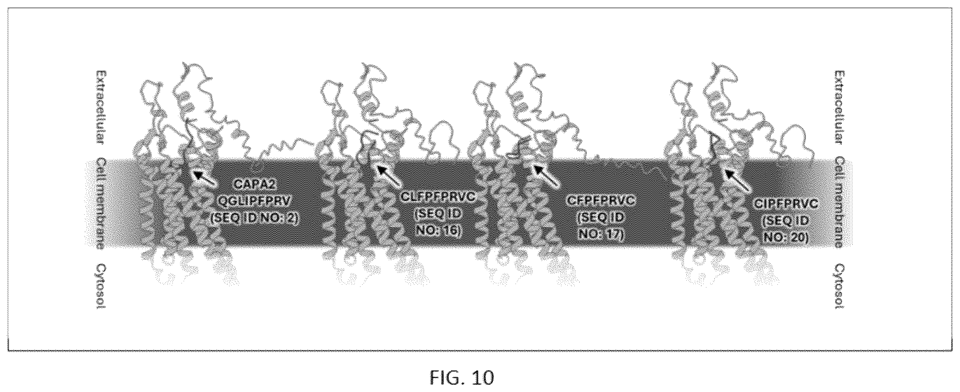

Predicted ligand binding domains of CAPA-R, a GPCR, were identified using 3D structural models generated with ColabFold. The binding locations of CAPA-R for each of the three selected terminally cyclized peptides (CLFPFPRVC (SEQ ID NO: 16), CFPFPRVC (SEQ ID NO: 17), and CIPFPRVC (SEQ ID NO: 20)) were similar to the active site occupied by the native ligand CAPA2 (QGLIPFPRV) binds. However, as shown in FIG. 10, slight differences in binding position were observed within the extracellular domain. Internally cyclized peptides were also modeled, but were not found to exhibit a different trend, and so are not shown.

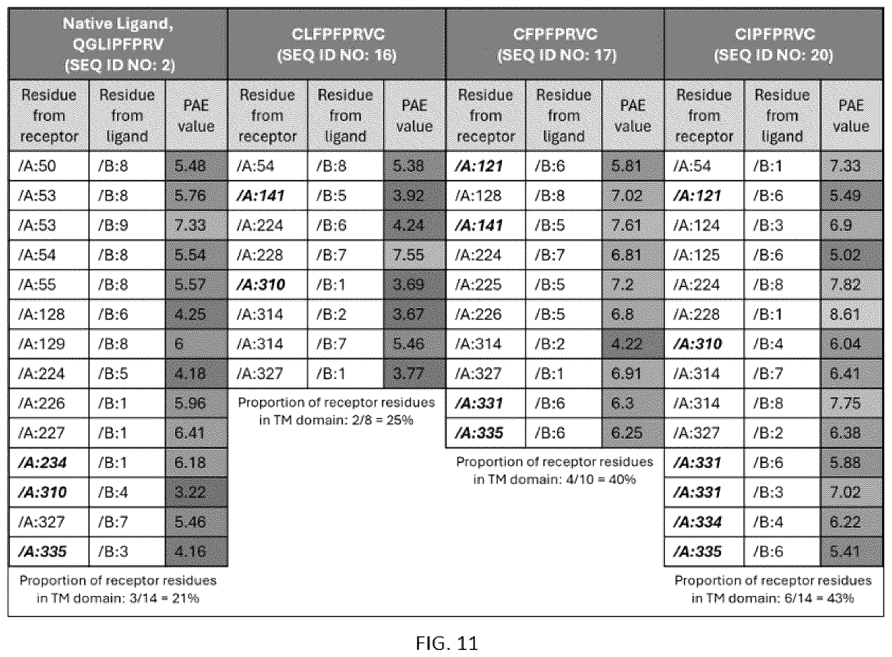

As shown in FIG. 11, the predicted aligned error (PAE) values for CAPA2 (QGLIPFPRV) bound to CAPA R ranged from 3.22 to 7.33, indicating relatively high confidence in the predicted ligand binding region. The cyclized peptide CLFPFPRVC (SEQ ID NO: 16) showed a similar PAE range (3.67-7.55), suggesting a confidence level comparable to that of CAPA2 in terms of protein domain proximity. The other two peptides, FPFPRVC (SEQ ID NO: 17) and CIPFPRVC (SEQ ID NO: 20), exhibited slightly higher PAE ranges (4.22-7.61 and 5.02-8.61, respectively), but these values still fall within a confidence range consistent with reliable domain interaction predictions.

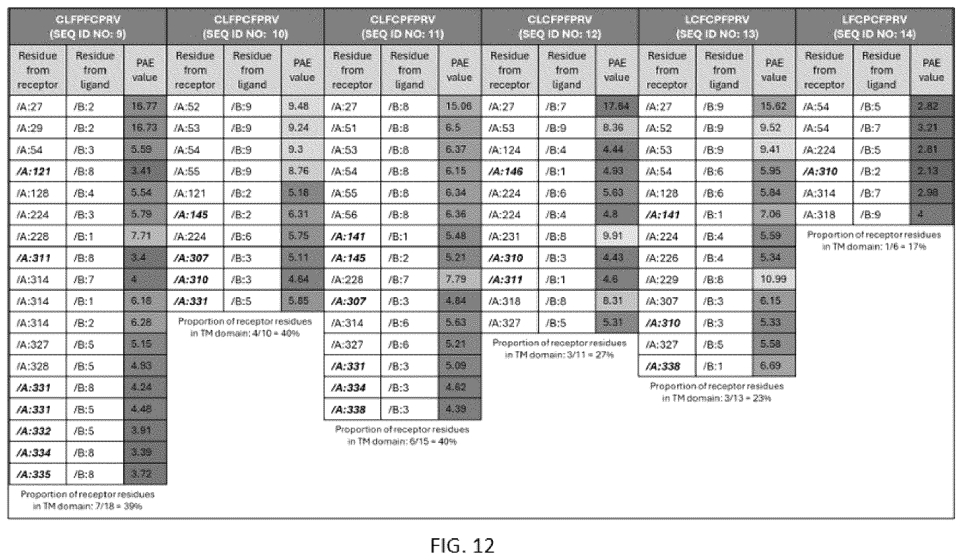

As shown in FIG. 12, PAE values of the six internally cyclized peptides (CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14)) ranged a little higher, from 3.4-16.77, 4.64-9.48, 4.39-15.06, 4.43-17.64, 5.33-15.62, and 2.81-4, for internally cyclized peptides, 1-5, 1-4, 1-3, 2-4, and 3-5, respectively.

### DISCUSSION

We selected six shortened analogs (6-8 aa): FPFPRV (SEQ ID NO: 5), LFPFPRV (SEQ ID NO: 4), and GLFPFPRV (SEQ ID NO: 3) derived from CAPA1 (EVQGLFPFPRV) (SEQ ID NO: 1), and IPFPRV (SEQ ID NO: 8), LIPFPRV (SEQ ID NO: 7), and GLIPFPRV (SEQ ID NO: 6) derived from CAPA2 (QGLIPFPRV) (SEQ ID NO: 2). With the exception of IPFPRV (SEQ ID NO: 8), these shortened linear analogs strongly activated CAPA-R and in some cases were more effective than the native ligand, indicating agonist-like activity in both the present disclosure and our previous work (Parks et al., 2025).

The two native ligands of F. occidentalis CAPA-R, CAPA1 (11 aa) and CAPA2 (9 aa), are linear, hydrophilic peptides. These properties likely reduce their stability in the highly acidic insect midgut, where peptide-degrading enzymes are abundant. In addition, their hydrophilicity may limit passage across the midgut barrier into the hemolymph. To address these limitations, two main strategies can be considered: encapsulation in hydrophobic nanoparticles, such as lipid-based carriers, and structural or chemical modification, including cyclization through disulfide bond formation to enhance proteolytic stability. Small peptide molecules may also be advantageous as control agents because they may penetrate the thrips gut or epidermis more readily. Our current findings provided the basis for the design and evaluation of cyclized CAPA analogs in WFT.

Since the first backbone cyclized peptide antagonists derived from moth PBAN were reported, more than a dozen studies have described cyclic analogs of PK/PBAN and CAPA peptides with improved bioavailability and potential application as insecticidal agents. In accordance with one or more aspects of the present disclosure, we designed two classes of cyclized peptides containing two cysteines positioned either internally or terminally and evaluated their ability to activate CAPA-R.

As shown in FIGS. 2B and 4B, among the six internally cyclized peptides, CLCFPFPRV (SEQ ID NO: 12), LCFCPFPRV (SEQ ID NO: 13), and LFCPCFPRV (SEQ ID NO: 14) strongly activated CAPA-R at both 500 nM and 1 μM and therefore displayed agonist-like activity, whereas CLFPFCPRV (SEQ ID NO: 9) and CLFCPFPRV (SEQ ID NO: 11) did not. To further examine agonistic properties, we used a dual-addition assay in which each peptide was first applied to CAPA-R and incubated for 1.5 min, a duration sufficient for receptor activation, followed by CAPA2 as a second challenge to assess receptor responsiveness. Based on these activity profiles, the six internally cyclized peptides could be divided into two functional groups.

The first group, consisting of CLFPFCPRV (SEQ ID NO: 9), CLFPCFPRV (SEQ ID NO: 10), and CLFCPFPRV (SEQ ID NO: 11), did not exhibit strong agonistic activity. These peptides induced only weak activation of CAPA-R upon initial application, followed by a response after CAPA2 addition. We hypothesize that the (F)PRV motif contributes to CAPA-R binding and mediates limited agonistic activity; however, this short 3-4 aa motif alone is likely insufficient to achieve full receptor activation. These peptides therefore appear to possess both partial agonistic and partial antagonistic properties.

The second group, consisting of CLCFPFPRV (SEQ ID NO: 12), LCFCPFPRV (SEQ ID NO: 13), and LFCPCFPRV (SEQ ID NO: 14), exhibited behavior more similar to that of the linear peptides and showed clear agonistic activity in the dual-addition assay. Once CAPA-R was strongly activated by these peptides, immediate re-stimulation with the native ligand did not produce the same level of activation. This observation suggests that CAPA-R may not have fully recovered from the initial stimulation and may require additional time to recover from desensitization. Upon agonist binding, CAPA-R undergoes a conformational change that activates heterotrimeric G proteins, resulting in increased cytosolic Ca² through both plasma membrane influx and release from intracellular stores. If the reduced response to CAPA2 reflects incomplete receptor recovery, these short peptides may be particularly promising agonists of thrips CAPA-R.

In contrast, as shown in FIGS. 2C and 4C, cyclized peptides containing a terminal disulfide bond exhibited weak or minimal activation of CAPA-R. In particular, CFPFPRVC (SEQ ID NO: 17), CGLIPFPRVC (SEQ ID NO: 18), CLIPFPRVC (SEQ ID NO: 19), and CIPFPRVC (SEQ ID NO: 20) produced minimal receptor activation during the initial challenge, whereas CAPA-R activation was almost fully restored after subsequent application of CAPA2. These findings suggest that terminal cyclization through a disulfide bond interferes with CAPA-R activation. One possible explanation is that the cyclic structure prevents the peptide from fitting properly within the receptor active site; alternatively, these peptides may interact weakly or bind to nonactive sites. Comparison of receptor activation profiles clearly distinguished internally from terminally cyclized peptides. Whereas some internally cyclized peptides retained agonistic activity, terminally cyclized peptides displayed neither clear agonistic nor antagonistic effects on CAPA-R. Because GPCR ligand-binding pockets are typically shaped by structurally complex extracellular loop regions, the partial activation observed for some cyclic peptides may reflect incomplete occupancy of the active site or reduced binding affinity, depending on disulfide bond placement. Supporting this interpretation, several internally cyclized variants, including CLCFPFPRV (SEQ ID NO: 12), LCFCPFPRV (SEQ ID NO: 13), and LFCPCFPRV (SEQ ID NO: 14), activated CAPA-R similarly to the linear peptide LFPFPRV (SEQ ID NO: 4). Given their greater structural and metabolic stability relative to linear peptides, cyclized peptides may represent promising scaffolds for the development of insecticidal agents with agonistic or antagonistic activity.

To further evaluate the effects of peptide modification on CAPA-R activity, as shown in FIG. 5, we examined terminally cyclized peptides after varying incubation periods before challenging the receptor with its native ligand. CAPA-R responsiveness to CAPA2 gradually recovered as the interval between peptide applications increased, as reflected by increasing relative fluorescence. Together, these results suggest that cyclization may reduce receptor activation while enhancing peptide stability. Cyclization through disulfide bond formation may offer several advantages for biopesticide development. Enhanced chemical stability may improve practical utility under field-relevant conditions, where compounds are exposed to UV radiation, heat, microbial degradation, and metabolism by insect enzymes. Cyclic peptides have been shown to resist degradation by heat and peptidases, although not necessarily by near-UV light. At the same time, peptide-based insecticides are still expected to degrade under natural environmental conditions and therefore are unlikely to accumulate in ecosystems.

We next tested whether the linear and cyclized peptides exerted insecticidal effects in thrips. As shown in FIG. 7, initial feeding assays with adult females revealed no significant differences in mortality relative to controls. We therefore focused on adult males and found that several cyclized peptides significantly reduced male survival. As shown in FIGS. 8A-8D and FIGS. 9B-9C, across 25 feeding replicates, nine cyclized peptides, including six internally cyclized analogs (i.e., CLFPFCPRV (SEQ ID NO: 9), CLFPCFPRV (SEQ ID NO: 10), CLFCPFPRV (SEQ ID NO: 11), CLCFPFPRV (SEQ ID NO: 12), LCFCPFPRV (SEQ ID NO: 13), and LFCPCFPRV (SEQ ID NO: 14)) and three terminally cyclized analogs (i.e., CLFPFPRVC (SEQ ID NO: 16), CFPFPRVC (SEQ ID NO: 17), and CIPFPRVC (SEQ ID NO: 20)), consistently exhibited insecticidal activity against male thrips. This result is particularly noteworthy because our previous studies showed that expression levels of CAPA peptides (Yun et al., 2023) and CAPA-R (Parks et al., 2025) are significantly higher in adult males than in females. Together, these findings suggest that the CAPA signaling system in adult males may be more susceptible to disruption by ingested CAPA analogs.

In field populations, WFT adults typically exhibit female-biased sex ratios, although these can vary substantially. In our observations, the sex ratio of thrips captured on sticky traps was approximately 4:1 (personal communication). Thus, the practical significance of male-selective lethality remains to be determined. Nevertheless, this sex-biased response is biologically intriguing and warrants further investigation, particularly to determine whether CAPA peptides contribute to male-specific physiological functions or are associated with male haploidy.

GPCRs are challenging targets for X-ray crystallography because their membrane-bound nature often compromises stability during purification and crystallization. However, recent advances in molecular, biochemical, and computational methods have established in silico modeling of insect GPCRs as a useful approach for insecticide discovery.

In accordance with one or more aspects of the present disclosure, 3D modeling of CAPA-R-ligand complexes suggested that the cyclized peptides bind within the same pocket as the native ligand, CAPA2, while adopting distinct binding modes. These models also enabled visualization of structure-activity relationships for the most lethal terminally cyclized peptides and the internally cyclized peptides. Modeling predictions generated with moderate to high confidence further indicated that the cyclic peptides, like the native ligand, bind within the active site of CAPA-R, with the principal differences among ligands lying in their orientation within the binding pocket. It will be of interest to determine whether bioactive peptides with sequences unrelated to the native ligand, such as those generated by receptor interference are likewise constrained to bind within the active site. Taken together, the integration of molecular and biological assays of CAPA peptides and CAPA-R with 3D modeling of GPCR-ligand complexes may facilitate the development of next-generation insecticides for thrips.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. An insecticide composition for controlling an insect, the composition comprising an effective amount of cyclized peptide having SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof.

2. The composition of claim 1, further comprising an insecticide carrier.

3. The composition of claim 2, wherein the carrier is saline.

4. The composition of claim 1, further comprising one or more suitable propellants, carriers, diluents, adjuvants, preservative, dispersants, solvents, or emulsifying agents.

5. The composition of claim 1, wherein the cyclized peptide is internally cyclized peptide having SEQ ID NO: 9, SEQ ID NO: 10, SEQ ID NO: 11, SEQ ID NO: 12, SEQ ID NO: 13, SEQ ID NO: 14, or a combination thereof.

6. The composition of claim 1, wherein the cyclized peptide is terminally cyclized peptide having SEQ ID NO: 15 SEQ ID NO: 16, SEQ ID NO: 17, SEQ ID NO: 18, SEQ ID NO: 19, SEQ ID NO: 20, or a combination thereof.

7. The composition of claim 1, wherein the cyclized peptide has SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof.

8. The composition of claim 1, wherein the cyclized peptide has SEQ ID NO: 12.

9. The composition of claim 1, wherein the cyclized peptide has SEQ ID NO: 13.

10. The composition of claim 1, wherein the cyclized peptide has SEQ ID NO: 14.

11. The composition of claim 1, wherein the cyclized peptide has SEQ ID NO: 15.

12. The composition of claim 1, wherein the cyclized peptide has SEQ ID NO: 16.

13. The composition of claim 1, wherein the cyclized peptide has SEQ ID NO: 17.

14. The composition of claim 1, wherein the cyclized peptide has SEQ ID NO: 18.

15. The composition of claim 1, wherein the cyclized peptide has SEQ ID NO: 19.

16. The composition of claim 1, wherein the cyclized peptide has SEQ ID NO: 20.

17. The composition of claim 1, wherein the cyclized peptide is produced by a genetically modified plant or a genetically engineered microbe, to express the cyclized peptide.

18. A spray composition for controlling an insect, comprising:
a) an effective amount of the composition of claim 1,
b) a propellant.

19. A bait composition for controlling an insect, comprising:
a) an effective amount of the composition of claim 1,
b) one or more food materials;
c) optionally a phagostimulant.

20. A method for controlling an insect comprising contacting an insect or its environment with a biologically effective amount of a composition of claim 1, wherein the mortality of the insect increases.

21. The method of claim 20, wherein the insect is a Frankliniella ssp.

22. The method of claim 20, wherein the cyclized peptide has SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or a combination thereof.

23. The method of claim 22, wherein the insect is Frankliniella occidentalis.


SEQUENCE LISTING (21 sequences)

SEQ ID NO: 1 - protein, 11 aa, synthetic construct
EVQGLFPFPRV

SEQ ID NO: 2 - protein, 9 aa, synthetic construct
QGLIPFPRV

SEQ ID NO: 3 - protein, 8 aa, synthetic construct
GLFPFPRV

SEQ ID NO: 4 - protein, 7 aa, synthetic construct
LFPFPRV

SEQ ID NO: 5 - protein, 6 aa, synthetic construct
FPFPRV

SEQ ID NO: 6 - protein, 8 aa, synthetic construct
GLIPFPRV

SEQ ID NO: 7 - protein, 7 aa, synthetic construct
LIPFPRV

SEQ ID NO: 8 - protein, 6 aa, synthetic construct
IPFPRV

SEQ ID NO: 9 - protein, 9 aa, synthetic construct
  DISULFID 1..6: note = Internally cyclized peptide
CLFPFCPRV

SEQ ID NO: 10 - protein, 9 aa, synthetic construct
  DISULFID 1..5: note = Internally cyclized peptide
CLFPCFPRV

SEQ ID NO: 11 - protein, 9 aa, synthetic construct
  DISULFID 1..4: note = Internally cyclized peptide
CLFCPFPRV

SEQ ID NO: 12 - protein, 9 aa, synthetic construct
  DISULFID 1..3: note = Internally cyclized peptide
CLCFPFPRV

SEQ ID NO: 13 - protein, 9 aa, synthetic construct
  DISULFID 2..4: note = Internally cyclized peptide
LCFCPFPRV

SEQ ID NO: 14 - protein, 9 aa, synthetic construct
  DISULFID 3..5: note = Internally cyclized peptide
LFCPCFPRV

SEQ ID NO: 15 - protein, 10 aa, synthetic construct
  DISULFID 1..10: note = Terminally cyclized peptide
CGLFPFPRVC

SEQ ID NO: 16 - protein, 9 aa, synthetic construct
  DISULFID 1..9: note = Terminally cyclized peptide
CLFPFPRVC

SEQ ID NO: 17 - protein, 8 aa, synthetic construct
  DISULFID 1..8: note = Terminally cyclated peptide
CFPFPRVC

SEQ ID NO: 18 - protein, 10 aa, synthetic construct
  DISULFID 1..10: note = Terminally cyclized peptide
CGLIPFPRVC

SEQ ID NO: 19 - protein, 9 aa, synthetic construct
  DISULFID 1..9: note = Terminally cyclized peptide
CLIPFPRVC

SEQ ID NO: 20 - protein, 8 aa, synthetic construct
  DISULFID 1..8: note = Terminally cyclized peptide
CIPFPRVC

SEQ ID NO: 21 - protein, 6 aa, synthetic construct
PGVLRF